(19) United States
(12) Patent Application Publication
(10) Pub. No.: US 2014/0107462 A1
(43) Pub. Date: Apr. 17, 2014

(54) OBJECT INFORMATION ACQUIRING APPARATUS AND OBJECT INFORMATION ACQUIRING METHOD

(71) Applicant: Koichiro Wanda, Yokohama-shi (JP)

(72) Inventor: Koichiro Wanda, Yokohama-shi (JP)

(73) Assignee: CANON KABUSHIKI KAISHA, Tokyo (JP)

(21) Appl. No.: 14/122,315

(22) PCT Filed: Jul. 10, 2012
(86) PCT No.: PCT/JP2012/004439

§ 371 Date: Nov. 26, 2013

(30) Foreign Application Priority Data
Jul. 29, 2011 (JP) 2011-166675

(51) Int. Cl. A61B 5/00

(52) U.S. Cl. CPC A61B 5/0095; A61B 2576/00
USPC 600/407

(57) ABSTRACT

The object information acquiring apparatus of the present invention acquires characteristic information in an object by using an acoustic signal output from an acoustic detector including an element for receiving an acoustic wave which has propagated in the object, and includes a receiving area determination unit for obtaining a receiving area of receiving the acoustic wave by using information of a specified acquisition area for the characteristic information specified by a user, configuration information related to accuracy of the characteristic information to be acquired, and receiving condition information on receiving conditions of the acoustic wave, and a scanning control unit for controlling scanning of the acoustic detector by using information of the receiving area obtained by the receiving area determination unit.

### TECHNICAL FIELD

The present invention relates to an object information acquiring apparatus and an object information acquiring method.

### BACKGROUND ART

The research of an optical imaging apparatus (imaging apparatus) capable of obtaining information in an object such as a living subject by using light irradiated from a light source such as a laser is being actively pursued in the medical field. As one type of such optical imaging technology, there is Photoacoustic Tomography (PAT). Photoacoustic tomography is the technique of visualizing information related to the optical characteristic values inside an object based on the acoustic waves generated from the body tissues which absorbed the energy of light that propagated and diffused in the object due to the photoacoustic effect. As an example of acquiring information related to the optical characteristic values, there is a method of detecting the acoustic waves at a plurality of locations surrounding the object, and mathematically analyzing and processing the obtained signals.

Information such as the initial sound pressure distribution or the optical energy absorption density distribution resulting from the irradiation of light obtained with the foregoing technology can be used for identifying the location of malignant tumors associated with the growth of new blood vessels. While the description of the optical energy absorption density distribution is omitted in the ensuing explanation, such description shall be included in the explanation of the initial sound pressure distribution. The generation and display of a three-dimensional reconstructed image based on the foregoing initial sound pressure distribution are useful in comprehending the inside of body tissues, and are expected to be useful for diagnosis in the medical field. However, since this technology is still on a learning curve, the generation of images of favorable image quality with less noise and artifacts is being demanded.

Here, a photoacoustic effect is the phenomenon where, when an object is irradiated with pulsed light, an acoustic wave (compressional wave, typically an ultrasonic wave) is generated based on the volume expansion in an area having a high absorption coefficient in the object to be measured. The acoustic wave that is generated based on the volume expansion resulting from the irradiation of pulsed light is referred to as a “photoacoustic wave” in the present invention.

Generally speaking, with photoacoustic tomography, the initial sound pressure distribution generated by light irradiation can be completely visualized, in theory, if the time change of the acoustic waves can be measured with an ideal acoustic detector (wideband/point detection) at various points on the closed space surface (in particular a spherical measuring surface) surrounding the entire object. Moreover, even if it is not a closed space, if the object can be measured based on a cylindrical shape or a tabular shape, it is mathematically known that the initial sound pressure distribution generated by light irradiation can be substantially reproduced (refer to Non Patent Literature 1).

Following Formula (1) is a partial differential equation which is used as the basis of PAT, and is referred to as a “photoacoustic wave equation”. Upon solving this formula, it is possible to describe the acoustic wave propagation from the initial sound pressure distribution, and theoretically obtain how and where an acoustic wave is detected.

$$\begin{matrix}[ Math . 1 ] & \\ ( {∇}^{2} - \frac{1}{{c}^{2}} \frac{{\partial }^{2}}{\partial {t}^{2}} ) p ( r , t ) = - {p}_{0} ( r ) \frac{\partial δ ( t )}{\partial t} & ( 1 )\end{matrix}$$

Here, r is the position, t is the time, p(r, t) is the time change of sound pressure, p₀(r) is the initial sound pressure distribution, c is the acoustic velocity, and o(t) is the delta function representing the shape of the optical pulse.

Meanwhile, the image reconfiguration of PAT is to elicit the initial sound pressure distribution p₀(r) from the sound pressure p_{d}(r_{d}, t) obtained at the detection points, and is mathematically referred to as an inverse problem. The Universal Back Projection (UBP) which is representatively used as the image reconfiguration method of PAT is now explained. The inverse problem of obtaining p₀(r) can be correctly solved by analyzing the photoacoustic wave equation of Formula (1) on a frequency space. UBP is the representation of the foregoing results on a time domain. Ultimately, following Formula (2) is derived.

$$\begin{matrix}[ Math . 2 ] & \\ {p}_{0} ( r ) = - \frac{2}{{Ω}_{0}} ∇ \cdot {\int }_{{S}_{0}} {{n}^{⋒}}_{0}^{S} d {{S}_{0} [ \frac{{p}_{0} ( {r}_{0} , t )}{t} ]}_{t = \vert r - {r}_{0} \vert} & ( 2 )\end{matrix}$$

Here, Ω₀ is the solid angle of the overall measurement area s₀ relative to an arbitrary reconfiguration voxel (or point of interest).

In addition, when the formula is modified in an easy-to-understand manner, following Formula (3) is derived.

$$\begin{matrix}[ Math . 3 ] & \\ {p}_{0} ( r ) = {\int }_{{Ω}_{0}} b ( {r}_{0} , t = \vert r - {r}_{0} \vert ) \frac{d {Ω}_{0}}{{Ω}_{0}} & ( 3 )\end{matrix}$$

Here, b(r₀, t) is projection data, and dΩ₀ is the solid angle of the acoustic detector dS₀ relative to the arbitrary observation point P.

The initial sound pressure distribution p₀(r) can be obtained by performing back projection to the foregoing projection data according to the integration of Formula (3).

$$\begin{matrix}[ Math . 4 ] & \\ b ( {r}_{0} , t ) = 2 p ( {r}_{0} , t ) - 2 t \frac{\partial p ( {r}_{0} , t )}{\partial t} & ( 4 ) \\ d {Ω}_{0} = \frac{d {S}_{0}}{{\vert r - {r}_{0} \vert}^{2}} cos θ & ( 5 )\end{matrix}$$

Note that b(r₀, t) and dΩ₀ are represented by above Formula (4) and Formula (5). Here, θ is the angle formed by the acoustic detector and the arbitrary observation point P.

If the distance between the sound source and the measuring position is sufficient in comparison to the size of the sound source (far distance/close sound field), following Formula (6) is derived.

$$\begin{matrix}[ Math . 5 ] & \\ p ( {r}_{0} , t ) ≪ t \frac{\partial p ( {r}_{0} , t )}{\partial t} & ( 6 )\end{matrix}$$

Here, b(r₀, t) is represented by following Formula (7).

$$\begin{matrix}[ Math . 6 ] & \\ b ( {r}_{0} , t ) = - 2 t \frac{\partial p ( {r}_{0} , t )}{\partial t} & ( 7 )\end{matrix}$$

As described above, with the image reconfiguration of PAT, it is known that the projection data b(r₀, t) can be obtained by performing temporal differentiation to the detection signal p(r₀, t) obtained with the acoustic detector, and the initial sound pressure distribution p₀(r) can be obtained by performing back projection according to Formula (3) (refer to Non Patent Literature 1).

However, Formula (1) which is a photoacoustic wave equation that was used to obtain Formula (3) assumes “constant sound velocity,” “omnidirectional measurement,” “impulse-based photoexcitation,” “wideband acoustic wave detection,” “point acoustic wave detection” and “sampling of continuous acoustic waves”. Realistically, it is difficult to realize a device capable of satisfying the foregoing assumptions.

For example, with an actual object, it is difficult to detect the acoustic waves with the entire closed space surface surrounding the overall object. Moreover, in order to increase the measuring area of the acoustic waves, it is necessary to increase the size and number of elements of the acoustic detector, as well as increase the signal processing of the respective elements and the number of control units, and they lead to increased production costs. Under the foregoing circumstances, a practical imaging apparatus which uses the PAT technology is often configured as an apparatus which detects acoustic waves, by using a probe (acoustic detector) of a limited size, from a specific direction of the object.

As an example of this kind of apparatus, as disclosed in Patent Literature 1, proposed is an apparatus which uses photoacoustic tomography of a flat plate measurement system. With this photoacoustic tomography, an object sandwiched between flat plates is irradiated with light, and acoustic waves are detected by an acoustic detector disposed on the flat plate. Here, the number of light irradiations and the number of acoustic wave receptions may be a multiple number. There are also cases where acoustic signals based on multiple light irradiations and acoustic wave receptions are used, and cases where an average value of the respective values calculated based on the acoustic signals is calculated and used.

Without limitation to flat plates, there are also cases where acoustic waves are acquired by disposing a probe with a mobile mechanism on a flat surface in which the relative positional relationship with the object is evident such as a surface that is in contact with the object or parallel to the surface of the object, or on a curved surface. If the area on the foregoing surface which receives acoustic waves while recording information related to the position of the element disposed on the probe is referred to as a receiving area, image reconfiguration can also be performed by using the acoustic waves that were detected in the entire receiving area.

Here, a receiving area is an area which occupies the receiving surface of the probe on which the elements are disposed based on the movement of the probe, and, while the time is different, a receiving area is also an area in which the acoustic waves can be received by the elements of the probe. The receiving area is not limited to a flat surface, and can also be a curved surface based on the shape of the receiving surface of the probe or the position of the disposed elements.

When acoustic waves are received while moving the probe within the receiving area, the position of the elements of the probe will change. Nevertheless, when considering the acoustic wave detected by each element to be an acoustic wave that is detected at the position of the elements on the receiving area upon detection, such acoustic wave can be deemed an acoustic wave that was detected at the respective positions on the receiving area. In other words, the foregoing acoustic wave can be deemed and treated as an acoustic wave that was detected by the probe in which elements are disposed on the receiving surface of a receiving area size. The photoacoustic signals of the entire receiving area can be detected by moving the probe according to the irradiation position or irradiation time of light in the case of photoacoustic waves and gathering the acoustic signal groups that were detected at the position of the respective elements on the receiving area.

Note that the method of detecting acoustic waves by causing the flat plate probe to be in close contact with the object has the following advantages; namely, acoustic waves can be detected with less noise, fixation of the position of the object or probe or the control of movement of the probe is facilitated while repeating the detection process, and so on.

Here, a valid acoustic signal according to the directionality of the elements of the acoustic detector is explained.

The term “valid acoustic signal” as used in this specification refers to an acoustic signal based on acoustic waves detected at a practical value or higher which is defined based on the characteristics or sensitivity of the elements of the probe. Generally speaking, with an ultrasonic probe, considered valid is an acoustic wave from a sound source that falls within a conical range defined based on a directivity angle which becomes the sound pressure of ½ from the sound pressure of the central axis of the elements. Thus, in the implementation of the present invention also, when this kind of acoustic wave based on a sound source within a directionality range of the elements is defined by being deemed valid, an acoustic signal based on such an acoustic wave is explained as a valid acoustic signal. However, the directivity angle does not necessarily have to be limited to an angle in which the sound pressure becomes ½. In the explanation of this specification, an acoustic signal based on the detection of an acoustic wave which uses, as the sound source, a range that is defined by being deemed valid for each element according to the characteristics or sensitivity of each element is referred to as a valid acoustic signal, and the range that is thereby defined by the directivity angle is explained as the range of directionality of the element.

Upon detecting acoustic waves by moving the acoustic detector along the flat plate, the orientation of the probe relative to the object will be limited. In other words, acoustic waves cannot be detected from the entire periphery of the imaging target. Since signals based on acoustic waves detected at the position of the elements on the receiving area of the probe having a limited orientation relative to the object are used, the result is an image reconfiguration based on an acoustic signal group that differs from the assumption of the photoacoustic wave equation.

In addition, if the imaging target area is considered the image reconfiguration area; that is, the reconfiguration area, conditions of the number of valid acoustic signals and the positions relative to the detecting positions will not necessarily be the same conditions at the respective points of interest of the reconfiguration area. In other words, there will be an area in the reconfiguration area having different conditions concerning the valid acoustic signals, and this will result in an area having a different image quality of the reconstructed image due to the foregoing difference in conditions.

With respect to the display of ultrasonic images, for instance, Patent Literature 2 discloses an example of determining the signal strength for each element of the probe, and displaying a map of the elements having a signal strength of a predetermined value or higher, or displaying a C-mode image if a three-dimensional image is generated. However, Patent Literature 2 displays images by determining the strength of the sent and received ultrasonic signals. Patent Literature 2 does not describe a method that can identify the areas having different image qualities in the reconstructed image of the photoacoustic waves, and does not disclose an imaging method which gives consideration to the difference in the image qualities in the reconstructed image of the photoacoustic waves.

### CITATION LIST

### Patent Literature

### [PTL 1]

U.S. Pat. No. 5,840,023

### [PTL 2]

Japanese Patent Application Publication No. 2007-312980

### Non Patent Literature

### [NPL 1]

PHYSICAL REVIEW E 71, 016706 (2005)

### SUMMARY OF INVENTION

### Technical Problem

Here, with conventional technology, the acoustic wave used for reconfiguration processing at one point (point of interest) in the reconfiguration area to be subject to reconfiguration processing is limited to the acoustic wave that is detected at the element position on the foregoing receiving area. Nevertheless, depending on the position of the elements on the receiving area and relative positional relationship relative to the point of interest, there are cases where a valid acoustic wave cannot be detected in the reconfiguration processing. This problem is explained below.

As described above, the acoustic signals detected by the elements of the probe are treated as valid acoustic signals when the sound source exists in a range that falls within a range based on the directionality of each element. Thus, in image reconfiguration, a valid acoustic signal group relative to the respective points of interest is extracted based on the detecting position information of the acoustic signals on the receiving area.

For example, if the receiving area is a flat surface, with a foot of a perpendicular lowered from the point of interest to the receiving area as the center, the extended line of the outer edge of the directivity angle and the surface resulting from extending the surface of the receiving area will intersect, and a circular area is thereby defined. This area is the acoustic wave detection area as the area in which elements for detecting the valid acoustic waves may exist. The number of valid acoustic signals will increase at points of interest in which the acoustic wave detection area is positioned to fall within the receiving area.

Nevertheless, since the acoustic wave detection area relative to the points of interest positioned near the end of the receiving area will not fall within the receiving area, the number of valid acoustic signals will be limited, and the image quality will thereby deteriorate.

Moreover, even if the number of valid acoustic signals is the same at a plurality of points of interest, if the detecting positions of the acoustic signals for use in reconfiguration are of a biased orientation, artifacts tend to be generated in a biased direction. This is because, since the image reconfiguration processing is performed using an acoustic signal group received by elements positioned asymmetrically, the difference with the assumption of the photoacoustic wave equation will increase.

Thus, with image configuration of PAT to which conventional technology is applied, artifacts tend to be generated when the sound source of photoacoustic waves is positioned on the normal of an end which veers off from near the center of the receiving area. Even greater artifacts are generated in the reconstructed image of the sound source of photoacoustic waves positioned near the outer edge of the receiving area or on the normal from a parallel surface that is even further outside of the receiving area.

Accordingly, a reconstructed image that is reconfigured using the acoustic signal group detected at a position which does not coincide with the assumption of the photoacoustic wave equation will contain points (points of interest) of a different image quality, and form an area having a different image quality in the image. If there is an area with a different image quality in the reconstructed image upon diagnosing the image generated with the imaging apparatus, the reliability of the diagnostic imaging will deteriorate, or otherwise increase the operation time of diagnostic imaging.

Generally speaking, with a medical imaging apparatus, the imaging time is desirably short since imaging imposes drain on the subject. Nevertheless, if there is not much restriction on the imaging time depending on the condition of the subject, an output image having a uniform image quality is more suitable for diagnostic imaging even if more imaging time is required.

Thus, while it is desirable that the acquisition of acoustic waves in the specified imaging area is completed within a short period of time, it is necessary to increase the receiving area of the acoustic waves upon considering the image quality of the area to be reconfigured. In other words, it is necessary to change the area to be scanned using the probe according to the image quality of the image to be reconfigured. However, if much time is required for setting the imaging area or the receiving area of acoustic waves, the burden on the subject may increase.

With an imaging apparatus for acquiring acoustic waves according to conventional technology, it was not possible to control the probe scanning of the specified imaging area according to the conditions of the acoustic waves that affect the image quality of the reconstructed image. Moreover, with an imaging apparatus in which a large receiving area is constantly set as the imaging area, there is a possibility that acoustic waves will be received from an area that is not required for the image reconfiguration, and unneeded burden will be imposed on the subject. Thus, desired is a method of promptly determining the receiving area according to the imaging area and the required image quality.

In light of the above, demanded is the provision of an imaging method capable of easily determining the appropriate acoustic wave acquiring method and performing imaging based on the specification of the image quality of the reconstructed image that is generated after the acoustic waves are acquired.

The present invention was devised in view of the foregoing problems, and an object of this invention is to provide technology for acquiring acoustic waves based on the setting of the image quality of the reconstructed image and the specification of the imaging area.

### Solution to Problem

The present invention adopts the following configuration. Specifically, the present invention is an object information acquiring apparatus which acquires characteristic information in an object by using an acoustic signal output from an acoustic detector including an element for receiving an acoustic wave which has propagated in the object, and includes a receiving area determination unit for obtaining a receiving area of receiving the acoustic wave by using information of a specified acquisition area for the characteristic information specified by a user, configuration information related to accuracy of the characteristic information to be acquired, and receiving condition information on receiving conditions of the acoustic wave, and a scanning control unit for controlling scanning of the acoustic detector by using information of the receiving area obtained by the receiving area determination unit.

The present invention additionally adopts the following configuration. Specifically, the present invention is an object information acquiring method by an object information acquiring apparatus which acquires characteristic information in an object by using an acoustic signal output from an acoustic detector including an element for receiving an acoustic wave which has propagated in the object, and includes a receiving area determination step of a receiving area determination unit obtaining a receiving area of receiving the acoustic wave by using information of a specified acquisition area for the characteristic information specified by a user, configuration information related to accuracy of the characteristic information to be acquired, and receiving condition information on receiving conditions of the acoustic wave, and a scanning control step of a scanning control unit controlling scanning of the acoustic detector by using information of the receiving area obtained in the receiving area determination step.

### Advantageous Effects of Invention

According to the present invention, it is possible to provide technology for acquiring acoustic waves based on the setting of the image quality of the reconstructed image and the specification of the imaging area.

Further features of the present invention will become apparent from the following description of exemplary embodiments with reference to the attached drawings.

### BRIEF DESCRIPTION OF DRAWINGS

FIG. 1 is a diagram showing the functional blocks of the object information acquiring apparatus in the embodiments.

FIG. 2 is a diagram showing the configuration upon realizing, with software, the information processing unit in the embodiments.

FIG. 3 is a diagram showing a configuration example of the acoustic signal measurement unit in the embodiments.

FIG. 4 is a flowchart showing the processing from starting the imaging process to instructing the acoustic wave acquisition in the embodiments.

FIG. 5 is a diagram showing the relation among the object and the imaging area and the receiving area in the embodiments.

FIG. 6 is a flowchart showing the processing of the acoustic wave measurement in the embodiments.

FIG. 7 is a flowchart showing the processing from reconfiguration to display in the embodiments.

### DESCRIPTION OF EMBODIMENTS

The present invention is unique in that the receiving area of the acoustic waves is obtained by using information of the imaging area (specified acquisition area) specified by the user, and configuration information related to the image quality (accuracy of characteristic information). The preferred embodiments of the object information acquiring apparatus and the object information acquiring method according to the present invention are now explained in detail with reference to the appended drawings. However, the scope of the invention is not limited to the illustrated examples.

### Embodiments

The object information acquiring method according to this embodiment is an imaging method of generating a three-dimensional reconstructed image by obtaining an initial sound pressure distribution from detected acoustic waves. Moreover, the object information acquiring apparatus is an imaging apparatus for conducting diagnosis by using the reconfigured image, and in this embodiment a photoacoustic wave diagnostic apparatus is explained as an example. A photoacoustic wave diagnostic apparatus detects acoustic waves that were generated by irradiating light, and generates a three-dimensional reconstructed image based on information related to the detected acoustic waves.

Note that the term “imaging” as used in the present invention refers to receiving acoustic waves which have propagated from within an object, and acquiring characteristic information of that object for imaging the inside of the object. As the characteristic information, considered may be information which reflects the sound pressure of acoustic waves, the optical energy absorption density derived from the sound pressure, or object information such as the absorption coefficient or concentration of the substance configuring the tissue, and so on. Concentration of the substance is, for example, the oxygen saturation or oxy/deoxy hemoglobin concentration. Moreover, the characteristic information may also be acquired as numerical value data, or as image data showing the distribution information (characteristic information distribution) of the respective positions (respective points of interest) in the object. In other words, the characteristic information may also be acquired as image data showing the distribution information which reflects the absorption coefficient distribution, the oxygen saturation distribution or the like in the object.

In this embodiment, a plurality of acoustic signal groups in which the number and detection position of the acoustic signals are different are extracted from the acoustic signals detected in a single imaging process, and a plurality of three-dimensional reconstructed images are generated thereby. Thereupon, the imaging apparatus is controlled so that the conditions such as the number and position of the acoustic signal groups to be used for calculating the characteristic information at the respective points of interest in the specified imaging area become uniform. Moreover, acoustic wave reception information which affects the image quality of the reconstructed image at the respective points of interest is acquired. Subsequently, the receiving area of the acoustic wave is obtained so that image data can be generated with the set image quality, and reconfigured image data having a uniform image quality is generated.

Note that the foregoing “single imaging process” refers to the processing of acquiring the characteristic information in the specified imaging area, and there are cases where multi light irradiations and acoustic wave detections are carried out during a single imaging process. Moreover, an “imaging area” is a user-specified three-dimensional specification area (specified acquisition area) from which the characteristic information is to be acquired.

Moreover, “image quality” of the reconstructed image in the present invention refers to the accuracy of the acquired characteristic information (that is, reconfigured numerical value data and the like) based on the number of acoustic signals relative to the points of interest as the subject of reconfiguration, distance to the elements, positional bias of the elements, and so on. This accuracy also includes the accuracy of the correlation of the amount of characteristics of the image and the amount of characteristics in the object when generating image data having characteristics that correlate to predetermined characteristics in the object based on all or a part of the acquired characteristic information.

### (Schematic Functional Block Diagram)

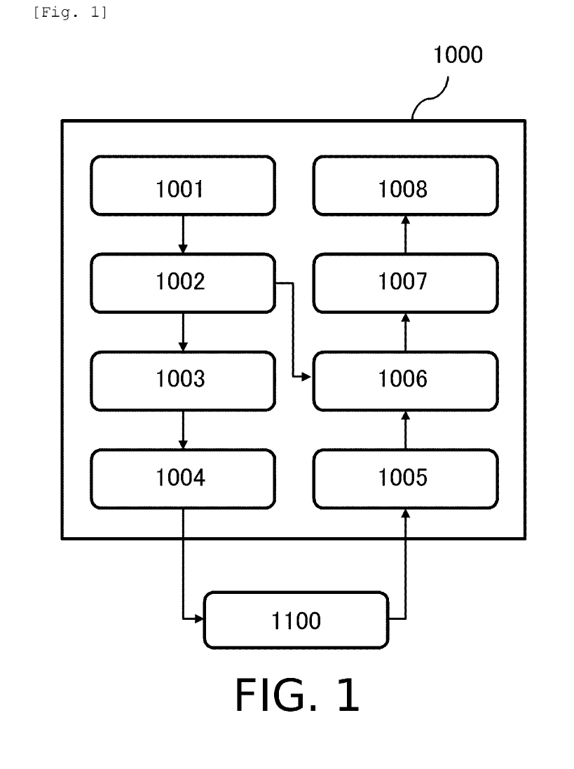

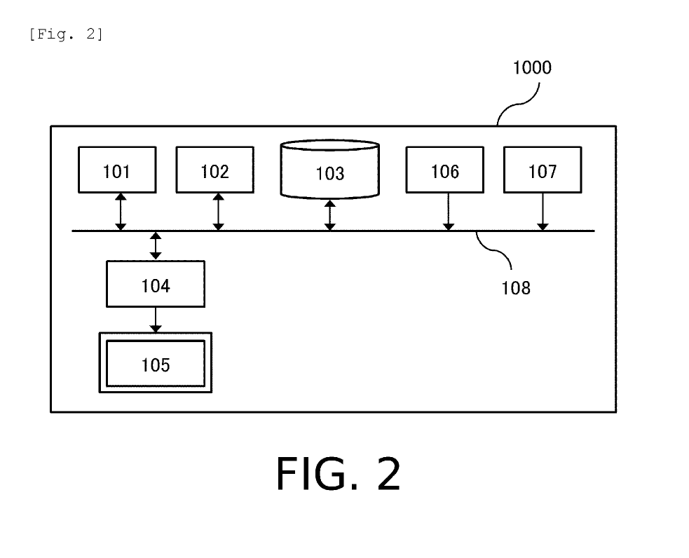

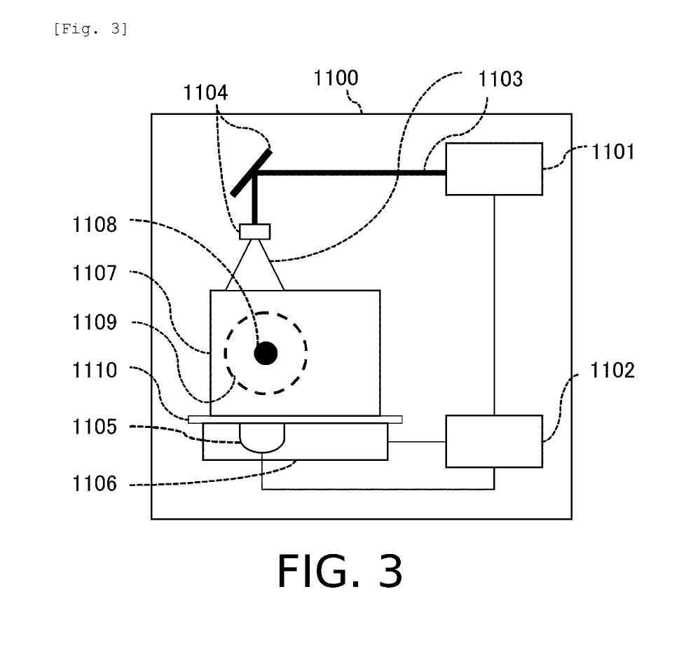

FIG. 1 shows the functional configuration of the photoacoustic wave diagnostic apparatus according to this embodiment. As shown in FIG. 1, the photoacoustic wave diagnostic apparatus according to this embodiment is configured from an information processing unit 1000, and an acoustic signal measurement unit 1100. Examples of the equipment configuration for implementing the respective functional blocks are shown in FIG. 2 and FIG. 3. FIG. 2 shows an example of the equipment configuration for implementing the information processing unit 1000 of the photoacoustic wave diagnostic apparatus. Moreover, FIG. 3 shows an example of the equipment configuration for implementing the acoustic signal measurement unit 1100.

The acoustic signal measurement unit 1100 is a block for measuring acoustic signals. The acoustic signal measurement unit 1100 controls the measurement of acoustic waves based on the acoustic wave acquisition instruction information instructed from the information processing unit 1000, and sends, to the information processing unit 1000, acoustic wave reception information based on the acoustic waves detected by the respective elements of the acoustic detector 1105.

The acoustic detector 1105 is, for example, a probe for detecting ultrasonic waves. Moreover, acoustic wave reception information includes at least an acoustic signal as a detection signal that was output by an element of the acoustic detector upon detecting (receiving) an acoustic wave, and receiving condition information as conditions concerning the reception of acoustic waves. Receiving condition information includes information related to the position of the elements disposed on the receiving surface of the acoustic detector 1105 as well as the sensitivity and directionality of such elements, and information related to the conditions upon receiving acoustic waves such as imaging parameters and other measurement information upon acquiring acoustic waves.

When the acoustic signal measurement unit 1100 causes the acoustic detector to perform scanning while moving the acoustic detector and thereby detects acoustic waves at the respective positions to which the acoustic detector had moved, the total scanned area is used as the receiving area, and the position of the elements that detected acoustic waves is treated as the element position on the receiving area. In the foregoing case, the position of the receiving area in the coordinate system within the apparatus and the information related to the element position on the receiving area are also included in the generated receiving condition information.

In addition, with the photoacoustic wave diagnostic apparatus, the control of the light source of light for generating acoustic waves and the information related to compression upon compressing the object are also included in the receiving condition information as the conditions upon acquiring acoustic signals.

Here, among the receiving condition information, information related to the position of elements may be any type of information so as long as it is information capable of identifying the positional relationship of the object or reconfiguration area to be subject to image reconfiguration, and the respective elements. For example, the foregoing information related to the position of elements may be information such as the number of respective elements, pitch of the arrangement of elements, and element position on the receiving surface, position of the acoustic detector, position and size of the object, position and size of the reconfiguration area to be subject to image reconfiguration, relative positional relationship thereof, and so on. In particular, when the acoustic detector is moved, the elements on the receiving surface of the acoustic detector move and detect the acoustic waves. While a plurality of acoustic waves are detected in a time series by each element, these acoustic waves may also be treated as information of each element position on the receiving area by determining the representative value for each detecting position on the receiving area.

Among the acoustic wave reception information, as the information related to acoustic signals, the detected acoustic signals may be sent as is, or information of acoustic signals after performing correction such as the sensitivity correction or gain correction of the elements may be sent. Moreover, it is also possible to repeat multiple light irradiations to the same position and detections at the same element position on the receiving area in one imaging processing, and send the average of the obtained acoustic signals.

Note that the same element of the acoustic detectors does not necessarily have to detect the element position on the receiving area. Even when the acoustic detector is moving, so as long as an element having the same capacity detects the same position on the receiving area, the detected acoustic signals may be deemed to be the acoustic signals of the same position for each element position on the receiving area, and may be the subject of averaging.

Among the acoustic wave reception information, information that can also serve as a static constant according to the device configuration may be stored in a main memory 102 or a magnetic disk 103 of the information processing unit 1000 in advance, and read during the image reconfiguration processing. However, information which is dynamically set each time imaging is performed is sent, as a part of the acoustic wave reception information, from the acoustic signal measurement unit 1100 to the information processing unit 1000.

For example, considered is the information related to the position of elements on the receiving surface of the acoustic detector 1105. After the identifier of the elements and the position information of the acoustic detector are sent from the acoustic signal measurement unit 1100 to the information processing unit 1000, it is also possible to implement the present invention by referring to the relative positional information of the acoustic detector and the elements stored in the information processing unit 1000 in advance.

The functional blocks of the information processing unit 1000 are now explained with reference to FIG. 1.

The information processing unit 1000 acquires instructions from a user related to imaging, and determines the acoustic wave measuring method to be employed by the acoustic signal measurement unit 1100 in consideration of the configuration information of the image quality of the reconstructed image, and instructs the acoustic signal measurement unit 1100 to measure the acoustic signals based on the determined measuring method. The information processing unit 1000 additionally uses the acoustic wave reception information obtained from the acoustic signal measurement unit 1100 to perform three-dimensional image reconfiguration processing and display the data of the obtained image.

The information processing unit 1000 includes an imaging instruction information acquisition unit 1001, a reconfiguring method determination unit 1002, an acoustic wave measuring method determination unit 1003, an acoustic wave measuring method instruction unit 1004, an acoustic wave reception information acquisition unit 1005, a reconfiguration processing unit 1006, a display information generation unit 1007, and a display unit 1008. However, the display unit 100 may be prepared separately from the object information acquiring apparatus of the present invention.

The imaging instruction information acquisition unit 1001 acquires instruction information (imaging instruction information) related to imaging that was input by the user via an input unit 106 (refer to FIG. 2). As the instruction information related to imaging, included are at least information of the imaging area (information of specified acquisition area) which specifies the area to be imaged in the apparatus of the acoustic signal measurement unit 1100, and configuration information related to the image quality (information related to the acquired accuracy of characteristic information) for specifying the image quality of the reconstructed image.

The imaging area is a three-dimensional area based on the specification from the user, and is a specified acquisition area of characteristic information. Any specifying method may be used so as long as the specifying method enables the imaging apparatus to specify the area to be imaged. For example, the user may specify and input only the two-dimensional area on the object compressed with a compression plate. This two-dimensional area may also be referred to as a specified imaging area. Subsequently, the acoustic signal measurement unit 1100 may measure the thickness of the object in the imaging apparatus, and consider the specified imaging area as one face, and use, as the imaging area, the hexahedron having the measured thickness as the height.

Otherwise, it is also possible to adopt a method of storing the area specified with a coordinate system in the acoustic signal measurement unit 1100 as preset information in the information processing unit 100, and specifying the identifier of that area. In the foregoing case, the object is disposed according to the area that is preset in the acoustic signal measurement unit 1100.

Configuration information related to the image quality is configuration information related to the accuracy of characteristic information and, for instance, is the number of acoustic signals used in the reconfiguration processing. It is also possible to specify the number of acoustic signals used at the respective points of interest in the reconfiguration area, or the relative detecting position of the acoustic signals used at the respective points of interest relative to the points of interest. Moreover, the detecting position of the acoustic signals can also be specified as the position of a valid acoustic signal. The condition may also be such that the acoustic signals are gathered at all positions of the elements on the receiving area which could fall into the range of directionality of the elements of the acoustic detector. The size and bias of artifacts can be inhibited based on the foregoing conditions related to acoustic signals. In addition, depending on the reconfiguration algorithm, it is also possible to set and specify parameters according to the characteristics of the reconfiguration algorithm, or add the acoustic characteristics or receiving conditions of the environment to detect the acoustic waves. As the user's input, used may be an input method of selecting the image quality level in which the foregoing conditions are preset. Moreover, configuration information related to the image quality is not limited to the configuration information set based on the user's specification, and may also be information that is preset in the apparatus.

The imaging instruction information acquisition unit 1001 acquires imaging instruction information, and sends the acquired imaging instruction information to the reconfiguring method determination unit 1002.

The reconfiguring method determination unit 1002 determines the reconfiguring method and the reconfiguration area. The reconfiguration area is an area (acquirable area) capable of acquiring the characteristic information with the set image quality (accuracy) relative to the imaging area as the specified acquisition area. The appropriate reconfiguring method and reconfiguration area are determined based on the imaging area and image quality that were specified based on the imaging instruction information. Here, the reconfiguring method determination unit functions as the acquirable area determination unit. The reconfiguring method determination unit 1002 determines the reconfiguring method and reconfiguration area by using information, which is stored in advance in the main memory 102 or the magnetic disk 103, related to the capacity of the acoustic signal measurement unit 1100 and the reconfiguration processing capacity of the reconfiguration processing unit 1006.

The reconfiguring method determination unit 1002 generates information related to the determined reconfiguring method as the reconfiguration instruction information. As the reconfiguration instruction information, there is information related to the reconfiguration area, and information related to the reconfiguring method such as the parameters of the reconfiguring processing including the reconfiguration algorithm, number of voxels to be reconfigured, pitch, and other matters. If the acoustic wave measurement environment or reconfiguration in the imaging area cannot be performed based on the same processing conditions, it is also possible to adopt a method of partitioning the inside of the imaging area into a plurality of reconfiguration areas, and generating a plurality of types of reconfiguration instruction information of the respective areas.

The reconfiguring method determination unit 1002 sends the generated reconfiguration instruction information to the reconfiguration processing unit 1006. Moreover, the reconfiguration instruction information and the imaging instruction information are sent to the acoustic wave measuring method determination unit 1003. However, the imaging instruction information acquired by the acoustic wave measuring method determination unit 1003 may also be directly acquired from the imaging instruction information acquisition unit 1001.

The acoustic wave measuring method determination unit 1003 determines the acoustic wave measuring method of the acoustic signal measurement unit 110 based on the acquired reconfiguration instruction information and imaging instruction information. Moreover, the receiving area in the case of detecting acoustic waves in a broad range by scanning the acoustic waves with the acoustic detector is also determined. In other words, the acoustic wave measuring method determination unit 1003 calculates the receiving area required for generating the image data with the specified image quality at the respective points of interest of the reconfiguration area based on the information of the imaging area and the configuration information related to the image quality.

Here, with an acoustic detector which sends and receives ultrasonic waves based on a standard linear system, it is often the case that one face of a hexahedron area to be imaged is scanned. Nevertheless, the embodiments of the present invention are not limited to cases of scanning only one face of the hexahedron area to be imaged. Moreover, it is not necessary to determine the scanning range, which is dependent on the angle of departure of ultrasonic waves as with an acoustic detector of a sector system.

Moreover, the acoustic wave measuring method determination unit 1003 additionally determines the pitch of the positioning of the elements on the receiving area for the elements of the acoustic detector 1105 to detect the acoustic signals required for the reconfiguration processing. The parameters upon controlling the apparatus for detecting acoustic waves and the correction method based on the acoustic characteristics in the apparatus are basically implemented by the acoustic signal measurement unit 1100. However, conditions such as the parameters or correction method related to the acoustic wave acquisition conditions pertaining to the image quality of the reconfiguration processing may also be determined by the acoustic wave measuring method determination unit.

The acoustic wave measuring method determination unit 1003 generates acoustic wave acquisition instruction information required for the acoustic signal measurement unit 1100 to measure the acoustic signals based on the foregoing information, and sends the generated acoustic wave acquisition instruction information to the acoustic wave measuring method instruction unit.

Here, a case is explained where the acoustic wave acquisition instruction information is prepared for each imaging process, but it is also possible to adopt a method of preparing equivalent acoustic wave acquisition instruction information in advance, and selecting the acoustic wave acquisition instruction information. In the foregoing case, the acoustic wave measuring method determination unit sends the identifier of the acoustic wave acquisition instruction information prepared in advance to the acoustic wave measurement instruction unit.

The acoustic wave measuring method instruction unit 1004 sends the acoustic wave acquisition instruction information to the acoustic signal measurement unit 1100, and gives instructions for measuring acoustic waves. If the acoustic wave acquisition instruction information is information prepared in advance, the acoustic wave acquisition instruction information may also be read from the main memory 102 or the magnetic disk based on the identifier sent from the acoustic wave measuring method determination unit 1003.

The acoustic wave reception information acquisition unit 1005 acquires the acoustic wave reception information including the acoustic signals sent from the acoustic signal measurement unit 1100 that measured the acoustic waves according to the instructions. In addition, the acoustic wave reception information acquisition unit 1005 sends the acquired acoustic wave reception information to the reconfiguration processing unit 1006.

The reconfiguration processing unit 1006 performs three-dimensional image reconfiguration by using only the selected acoustic signals for each point of interest in the area to be subject to image reconfiguration, and generates a three-dimensional reconstructed image (volume data) based on the acoustic wave reception information.

The reconfiguration processing unit 1006 performs the reconfiguration processing based on the reconfiguration instruction information sent from the reconfiguring method determination unit 1002, and the acoustic wave reception information sent from the acoustic wave reception information acquisition unit 1005. Note that, if any change in configuration or correction is required in the reconfiguration area or the reconfiguration parameters due to reasons such as the unsuccessful measurement of acoustic waves of a certain area in the measurement of acoustic waves performed by the acoustic signal measurement unit 1100, such change or correction may be performed at this stage. In the foregoing case, if information related to the condition of the acoustic wave measurement is included in the acoustic wave reception information, the change or correction can be determined from the difference from the acoustic wave measurement that is anticipated by the reconfiguration instruction information.

Here, with the image reconfiguration processing to be performed by the reconfiguration processing unit 1006, embodiments of the present invention can be applied to both the time-domain method and the Fourier-domain method so as long as it is a three-dimensional image reconfiguration based on an analytical solution.

In the case of a photoacoustic wave diagnostic apparatus, a value showing the absorption coefficient distribution of light in the object is calculated, and a reconfigured three-dimensional image if thereby generated. Since the level of absorption of light in the object will differ according to the wavelength of the irradiated light, the difference in compositions in the object can be displayed as a three-dimensional image. Correction that is required when the intensity of light is not uniform in the reconfiguration area or other correction related to the reconstructed image may be performed at this stage.

The reconfiguration processing unit 1006 sends the generated reconstructed image to the display information generation unit 1007.

The display information generation unit 1007 generates display information from the reconstructed image received from the reconfiguration processing unit 1006. If the reconstructed image is a flat image and the range of value enables display at the brightness value of the display without any change, the display information can be used directly as the display information without requiring any special conversion. Even in cases where the reconstructed image is a three-dimensional image such as volume data, display information can be generated based on an arbitrary method such as volume rendering, multiplanar reconstruction, maximum intensity projection, or the like. Moreover, if the range of value of the voxel value is a range of value which exceeds the range of value of the brightness value of the display, processing is performed on Windows as needed, and display information is generated based on the image value that can be displayed with a display. The created display information may be display information containing information capable of displaying the reconstructed image. In other words, in order to display the reconstructed image simultaneously with other information, the information may be an integration of a plurality of pieces of information.

The display information generation unit 1007 sends the display information to the display unit 1008.

The display unit 1008 is a graphic card for displaying the generated display information and a display device such as a liquid crystal display or a CRT display, and displays the display information sent from the display information generation unit 1007. The display unit 1008 may be configured integrally with the imaging apparatus, or configured independently from the imaging apparatus.

Note that, in the explanation of the imaging apparatus to perform the image reconfiguring method of the present invention, the acoustic signal measurement unit 1100 and the information processing unit 1000 are explained separately. Specifically, an equipment configuration such as a measurement device or a control device (or PC) for digital mammography is indicated as an example. Nevertheless, the equipment configuration may be one object information acquiring apparatus including the acoustic signal measurement unit 1100 and the information processing unit 1000. For example, adopted may be a device configuration where a standard ultrasonic diagnostic apparatus is equipped with functions corresponding to the acoustic signal measurement unit 1100 and the information processing unit 1000 of the present invention.

### (Configuration of Information Processing Unit)

FIG. 2 is a diagram showing the basic configuration of a computer for realizing, with software, the functions of the respective units of the information processing unit 1000.

The CPU 101 mainly controls the operation of the respective constituent elements of the information processing unit 1000. The main memory 102 stores control programs to be executed by the CPU 101, and provides a working area when the CPU 101 executes the programs. The magnetic disk 103 stores an operating system (OS), device drivers of peripheral equipment, and various types of application software including programs for performing the processing of the flowcharts described later. The display memory 104 temporarily stores display data for the monitor 105.

The monitor 105 is, for example, a CRT display or a liquid crystal monitor, and displays images based on data from the display memory 1204. The input unit 106 is a mouse, a keyboard or the like to be used by the operator for performing pointing input to textual input. Operations by the operator in the embodiments of the present invention are performed using the input unit 106. However, while the input unit 106 and the monitor 105 may be provided within the object information acquiring apparatus of the present invention, the input unit 106 and the monitor 105 may also be configured independently from the object information acquiring apparatus. An I/F 107 is used for exchanging various data between the information processing unit 1000 and the outside, and is configured from IEEE1394, USB, Ethernet port (registered trademark) or the like. The data acquired via the I/F 107 is incorporated into the main memory 102.

Functions of the acoustic signal measurement unit 1100 are realized via the I/F 107. Note that the respective constituent elements described above mutually connected communicably via a common bus 108.

### (Configuration of Devices)

FIG. 3 is a diagram showing an example of the configuration of the acoustic signal measurement unit 1100.

The light source 1101 is a light source of irradiation light such as a laser or light-emitting diode for irradiating an object. As the irradiation light, used is irradiation light of a wavelength in which the level of absorption with a specific component, among the components configuring the object, is expected to be strong.

The control unit 1102 controls a light source 1101, an optical device 1104, an acoustic detector 1105, and position control means 1106. The control unit 1102 additionally amplifies the electric signal (acoustic signal) of the photoacoustic waves obtained with the acoustic detector 1105, and converts the analog signals into digital signals. Moreover, the control unit 1102 performs various types of signal processing and various types of correction processing. Moreover, the control unit 1102 sends acoustic signals from the acoustic signal measurement unit 1100 to, for instance, external equipment such as the information processing unit 1000 via an interface not shown.

The control unit 1102 additionally performs various types of control for synchronizing the signals of the photoacoustic waves detected by the acoustic detector 1105 with the timing of laser irradiation. In addition, the control unit 1102 also performs the signal processing of adding and averaging the acoustic signals for each element obtained by irradiating the laser a plurality of times, and calculating the average value of the acoustic signals for each element.

As the contents of laser control, there are the control of the timing, waveform, strength and so on of the laser irradiation. With respect to the control of the position control means 1106 of the acoustic detector, the position of the acoustic detector 1105 is moved to an appropriate position.

The optical device 1104 is, for example, a mirror which reflects light, or a lens which focuses, expands, or changes the shape of light. Any such optical component may be used so as long as the object 1107 can be irradiated with light 1103 emitted from the light source in the intended shape. A plurality of light sources 1101 and optical devices 1104 may be used in certain cases.

Note that the light 1103 irradiated from the light source 1101 is sometimes caused to propagate by using an optical waveguide or the like. As the optical waveguide, an optical fibre is preferably used. When a plurality of light sources and an optical fibre are used, it is also possible to use a plurality of optical fibres for the respective light sources, and thereby guide light to the surface of the living body. Otherwise, light from a plurality of light sources can be guided to a single optical fibre, and all light may be guided to the living body by using only one optical fibre.

When the object 1107 is irradiated, via the optical device 1104, with the light 1103 generated by the light source 1101 according to the foregoing configuration and based on the control of the control unit 1102, the optical absorber 1108 in the object absorbs light, and generates photoacoustic waves 1109. In addition, the generated photoacoustic waves 1109 propagate in the object and are discharged outside the object. In the foregoing case, the optical absorber 1108 corresponds to the sound source.

The acoustic detector 1105 is configured from a transducer which uses the piezoelectric phenomenon, a transducer which uses the resonance of light, a transducer which uses the change in capacity, or the like. Any acoustic detector may be used so as long as the acoustic detector can detect acoustic waves. The acoustic detector 1105 may detect acoustic waves by directly coming into contact with the object 1107, or, after compressing the object with plates such as the flat plates 1110, detect the photoacoustic waves 1109 from the compressed object through the flat plates.

The acoustic detector 1105 is explained on the premise that a plurality of elements (detection elements) are disposed two-dimensionally. As a result of using the foregoing two-dimensional array elements, acoustic waves can be detected simultaneously at a plurality of locations so as to shorten the detection time and reduce the influence from the vibration of the object or the like. Moreover, an acoustic impedance matching agent such as gel or water (not shown) for inhibiting the reflection of acoustic waves may be used between the acoustic detector 1105 and the object.

Here, the area where the object is irradiated with light or the acoustic detector 1105 may be movable. The optical device 1104 may be configured so that the light generated from the light source can move on the object. As methods of moving the area where the object is irradiated with light, there are a method using a movable mirror or the like, a method of mechanically moving the light source itself, and so on. In addition, the acoustic detector can be configured to be movable by providing a position control means 1106 for moving the position of the acoustic detector 1105. As an example of the position control means 1106, there is a method of moving the acoustic detector 1105 above the flat plate 1110, via a motor, based on information of the position sensor.

Control of the area to be illuminated with light and the position of the acoustic detector 1105 is performed by the control unit 1102.

Moreover, the control unit 1102 may also perform control so that the area where the object 1107 is irradiated with light (irradiated light) and the acoustic detector 1105 move in synch. As a result of the light irradiation area being movable, a broader range can be irradiated with light, and photoacoustic waves can be detected with the acoustic detector that is positioned appropriately relative to the irradiation area.

Here, as the method of moving the acoustic detector, any moving method may be adopted so as long as the acoustic signals detected with the elements at the respective positions on the receiving area can be deemed the acoustic signals detected with the acoustic detector having the elements at the foregoing positions. For example, if the surface where the elements of the acoustic detector are disposed is rectangular, after moving the acoustic detector in a distance corresponding to the vertical or horizontal size of the face, without any spacing in between, where the elements of the acoustic detector are disposed according to the moving direction of the acoustic detector, the acoustic detector may stand still at the respective positions to detect the acoustic waves.

As a result of treating the acoustic signal group as acoustic signals of each element position on the receiving area, the acoustic detector can be deemed an acoustic detector of a size in which elements in multiplication of the number of moved elements are disposed at the same element pitch in the moving direction of the acoustic detector.

Note that the travel distance of the acoustic detector does not necessarily have to be the same size as the size of the face on which the elements of the acoustic detector are disposed. Moreover, even in cases where the face where the elements are disposed is of a different shape, the travel distance of the acoustic detector may also be such that the area before movement and the area after movement of the face where the elements are disposed come into contact or overlap. In addition, the moving method of the acoustic detector may also be a moving method with gaps in the receiving area, and the present invention can also be implemented with this kind of method. For example, the acoustic detector may be moved such that, by extracting the representative value or calculating the average value of the acoustic signals of the respective positions on the receiving area that received the acoustic waves, the acoustic signals at the respective positions on the receiving area ultimately become uniform. In the foregoing case, it will suffice if the acoustic signals of the respective positions corresponding to the element pitch are uniform upon deeming the acoustic detector to be an acoustic detector having a receiving surface of the receiving area size.

In addition, the moving method of the acoustic detector may also be such that the acoustic detector continuously detects the acoustic waves while continuously moving without standing still. The acoustic signals at the respective positions on the receiving area are determined by extracting signals to become the representative value or the average value from the position of the acoustic detector and the acoustic signal group detected for each element position in the receiving area corresponding to the moving range of the acoustic detector. By adjusting the speed or moving range of the acoustic detector, the acoustic detector can be deemed an acoustic detector in which elements are disposed at an arbitrary pitch in the receiving area of an arbitrary size. Moreover, the scanning control is facilitated and the scanning time is shortened. Note that the apparent pitch of the elements of the acoustic detector; that is, the pitch of the element position for detecting the acoustic waves in the receiving area may be an arbitrary pitch that is different from the pitch of the elements disposed on the acoustic detector.

The imaging apparatus of the present invention receives instructions regarding the imaging area from the user via the input unit 106, and additionally acquires acoustic signals that are required for the image reconfiguration of the imaging area. The imaging area is a three-dimensional area specified for each imaging process. Normally, the area where an object can be imaged in the imaging apparatus is determined based on the specification of the imaging apparatus, and the user can specify the area within the given range.

The input method of imaging area may be any method so as long as it is a method where the acoustic signal measurement unit 1100 can specify the intended imaging area. It is also possible to input the coordinates of the respective apexes of the hexahedron or mathematical formula within the imageable range in the imaging apparatus. It is also possible to adopt a method of the user specifying a rectangular area with a mouse on the camera image that captured the object, and using, as the imaging area, an area obtained by projecting the foregoing area on the object and a three-dimensional area that is identified by measuring the depth direction of the object. In the foregoing case, the hexahedron to be used as the imaging area can be identified by imaging the camera image through the transparent flat plate, and measuring the thickness of the object from the flat plate. Note that a hexahedron area is generally used upon treating volume data as three-dimensional data, but it goes without saying that the imaging area does not necessarily have to be a hexahedron.

### (Processing Routine)

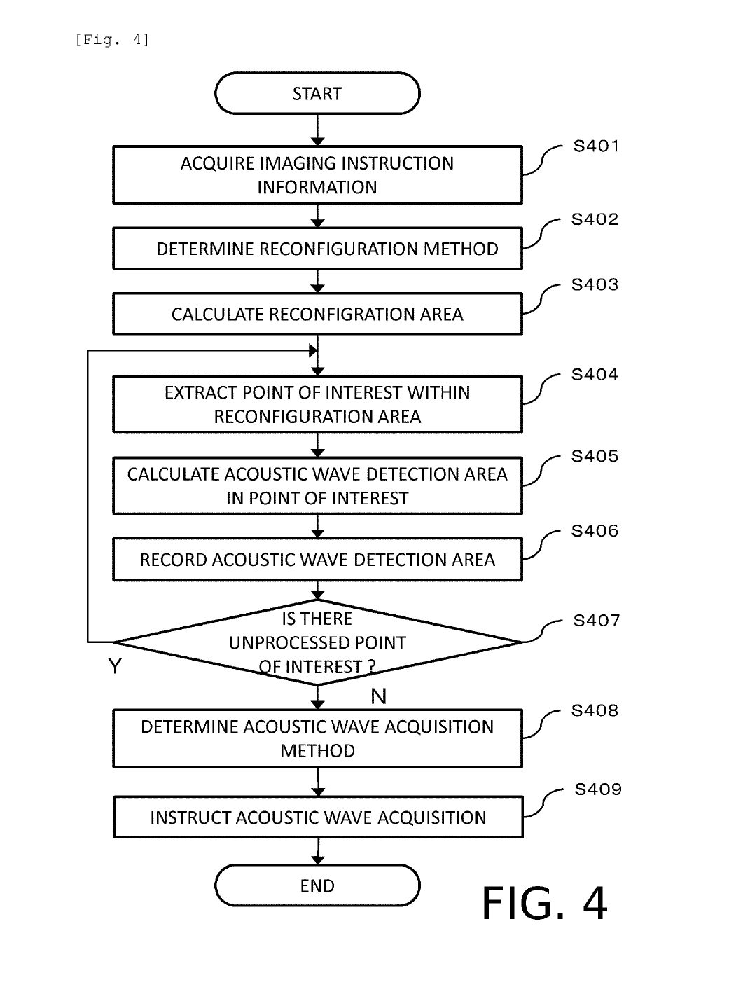

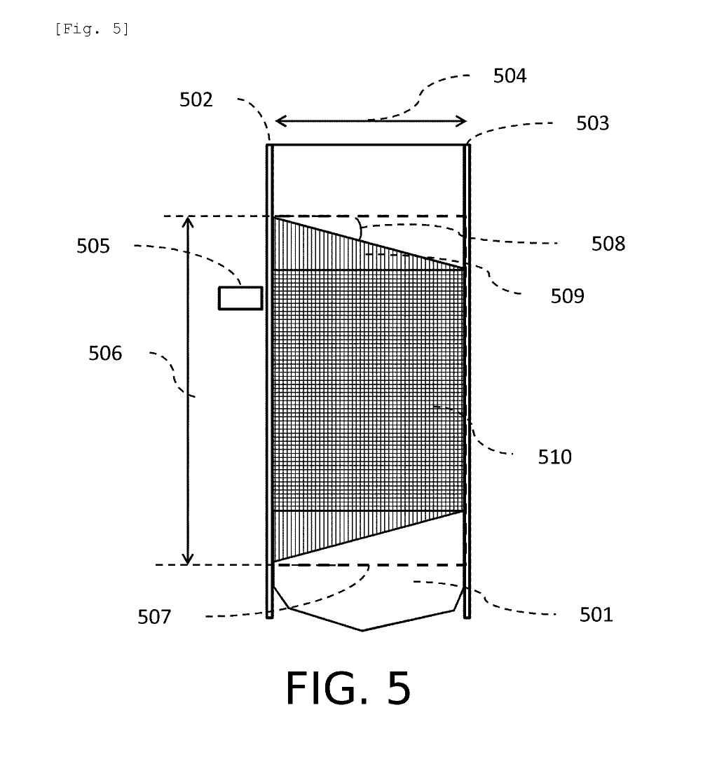

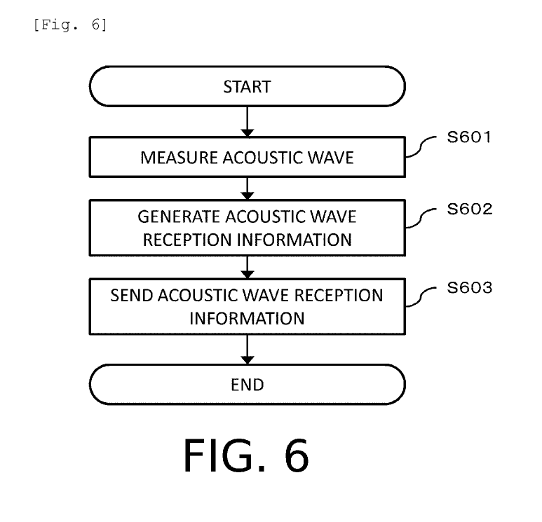

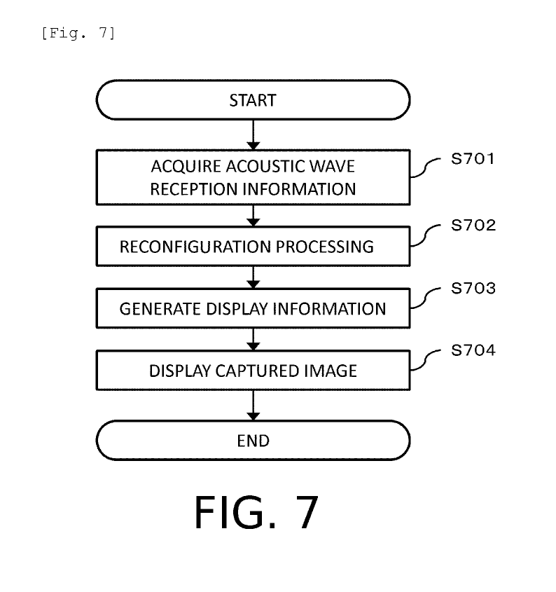

The processing routine of the imaging method according to an embodiment of the present invention is now explained with reference to the flowchart of FIG. 4, FIG. 6 and FIG. 7, and FIG. 5.

An example of executing the imaging processing based on the specified image quality and displaying the imaging data is now explained. To summarize, foremost, the information processing unit 1000 determines the appropriate reconfiguring method and acoustic wave acquiring method based on the imaging area, image quality, and respective parameters, and gives instructions to the acoustic signal measurement unit 1100. Subsequently, the acoustic signal measurement unit 1100 acquires the acoustic wave reception information based on the acoustic wave acquiring method instructed from the information processing unit 1000, and once again sends such acoustic wave reception information to the information processing unit 1000. Next, the information processing unit 1000 performs reconfiguration processing based on the acoustic signals of the received acoustic wave, and displays the imaging data of the imaging area.

FIG. 4 is a flowchart showing the routine of the information processing unit 1000 determining the reconfiguring method and the acoustic wave acquiring method and sending such information to the acoustic signal measurement unit 1100 after the user inputs the settings for the imaging process. In other words, this is the routine before the actual measurement of the object is started. In this embodiment, the photoacoustic wave diagnostic apparatus of FIG. 3 is used as the imaging apparatus. Nevertheless, the target to which the present invention is applied is not limited to a photoacoustic wave diagnostic apparatus. The present invention can be applied to any apparatus so as long as it can acquire acoustic waves and generate a reconstructed image based on the acquired acoustic waves.

The flowchart of FIG. 4 is started from the following situation. Foremost, the imaging operator as the user closely adheres the object (for instance, the subject's breast) to the holding plate, and fixes the object to the imaging position of the acoustic signal measurement unit 1100. Thereafter, the user sets parameters related to the imaging or intended image quality in the information processing unit 1000, and performs operations for instructing the start of the imaging process.

In step S401, the imaging instruction information acquisition unit 1001 acquires, as the imaging instruction information, the configuration information related to the imaging process, and the configuration information related to the image quality. As the configuration information related to the imaging process, there are imaging parameters related to the imaging area or acquisition of photoacoustic waves. As the configuration information related to the image quality, there is information related to the acoustic signals (number of signals used for reconfiguration).

In this embodiment, with respect to the image quality, instructions are given for generating a reconstructed image using the maximum number of acoustic signals that could be detected in the directionality range from the respective points of interest relative to the respective points of interest in the reconfiguration area.

Here, the maximum number of acoustic signals that could be detected in the directionality range from the respective points of interest refers to the maximum number of acoustic signals that could be detected in a predetermined area that is set in advance. A predetermined area is, for example, an area in which the receiving surface is partitioned by a circle formed from the intersection point of the line extending from the point of interest to be subject to reconfiguration along the directivity angle of the detection element and the receiving area. Based on the foregoing definition, when the acoustic detector is within the acoustic wave detection area, the point of interest will exist in the directionality range of the detection element. The center of this circular area becomes the foot of a perpendicular lowered from the point of interest to the receiving area. As described above, a predetermined area corresponding to the directionality range of the detection element when viewed from a certain point of interest is referred to as an acoustic wave detection area.

Since the detection elements are disposed in a predetermined interval in the acoustic detector, if the acoustic wave detection area can be defined, then the maximum number of acoustic signals that could be detected will also naturally be defined.

However, even if the positions of the elements on the receiving area are of a given interval, the maximum number of acoustic signals that could be detected will differ according to the distance between the point of interest and the receiving area. In other words, as the distance between the point of interest and the receiving area increases, the acoustic wave detection area will broaden, and the maximum number of acoustic signals that could be detected will increase. If the distance from the receiving area is the same and the predetermined area falls within the receiving area, the result will be the same number. Nevertheless, in this embodiment, regardless of the distance between the point of interest and the receiving area, the acquisition of the maximum number of acoustic signals relative to the respective points of interest is set as the image quality.

When reviewing the acoustic signals detected in the directionality range, when reconfiguration is performed using the acoustic signals at point-symmetric positions relative to the center of the circle, bias in the shape of artifacts is reduced, and the image quality of the reconstructed image is improved. However, in this embodiment, since the bias in the artifacts is also deemed to be reduced if the maximum number of acoustic signals can be detected in the directionality range, the area is determined to be an area with a favorable image quality.

The imaging instruction information acquisition unit 1001 sends the acquired imaging instruction information to the reconfiguring method determination unit 1002.

In step S402, the reconfiguring method determination unit 1002 determines the reconfiguring method based on the imaging instruction information, and the information related to the acoustic signal measurement capacity of the acoustic signal measurement unit 1100. As the information related to the acoustic signal measurement capacity, used may be the information that is stored in the main memory 102 or the magnetic disk 103 in advance.

As the information related to the acoustic signal measurement capacity, for example, used may be information related to the imageable area, information of the acoustic detector (travel speed or signal processing capacity), or information such as the laser irradiation interval of the light source. Information related to the imageable area is information such as the position or size of the imageable area in the apparatus, area that can be scanned by the acoustic detector, or range that laser can be irradiated in the case of photoacoustic waves.

The reconfiguring method determination unit 1002 determines the reconfiguring method that can be executed with the specified image quality when an imaging area included in the imaging instruction information is the reconfiguration area. Contents of the determined reconfiguring method are the algorithm and parameters of the reconfiguration processing, the correction method to be additionally performed such as optical distribution correction, and so on. The algorithm and parameters of the reconfiguration processing contain information for specifying the acquisition position of the acoustic waves required for the reconfiguration of the respective points of interest in the reconfiguration area with the specified image quality.

Here, information related to the acoustic signal measurement capacity does not necessarily have to be considered. The reconfiguration area may also be determined only with instructions related to the reconfiguration processing such as the algorithm and parameters. In the foregoing case, there may be cases where it is not possible to measure the acoustic signals of an appropriate range relative to the imaging area. In the foregoing case, the present invention can be implemented by changing the size of the imaging area, or treating an area in the reconfiguration area corresponding to the imaging area as an area in which the acoustic signals for achieving the specified image quality could not be collected.

In step S403, the reconfiguration area to be subject to the reconfiguration processing during the imaging process is calculated. Normally, the area corresponding to the imaging area (specified acquisition area of the characteristic information based on the user's input) is used as the reconfiguration area (acquirable area of characteristic information and generating area of image data). Nevertheless, there are cases where an area that is different from the imaging area is used as the reconfiguration area. Such cases include a case where it is determined that the entire imaging area and image quality specified in the imaging instruction information are incompatible, or a case where it is determined that the capacity of the apparatus is insufficient relation to the type of reconfiguration algorithm or parameters to be applied.

Moreover, in order to shorten the reconfiguration processing time and consequently the overall imaging processing time, an area excluding an area where it is evident that the image quality is inferior may be used as the configuration area within the specified imaging area.

When it is determined that it is necessary to change the specified imaging area based on the foregoing reasons, the reconfiguring method determination unit 1002 calculates the reconfiguration area to be actually subject to image reconfiguration in substitute for the imaging area, and thereafter generates information related to the calculated reconfiguration area.

The reconfiguring method determination unit 1002 sends the determined reconfiguration instruction information (information related to the reconfiguring method and information related to the reconfiguration area) to the reconfiguration processing unit 1006 and the acoustic wave measuring method determination unit 1003.

In step S404, the acoustic wave measurement area determination unit 1003 extracts one point in the reconfiguration area as the point of interest based on the information related to the reconfiguration area sent from the reconfiguring method determination unit 1002. When the reconfiguration area is to be treated as three-dimensional volume data, the voxel of an arbitrary position may also be extracted.

In step S405, the acoustic wave detection area in the extracted point of interest is calculated. Calculation of the acoustic wave detection area is performed by using the reconfiguring method determined by the reconfiguring method determination unit 1002, and information related to the acoustic signal measurement capacity of the acoustic signal measurement unit 1100. In this embodiment, the reconfiguration processing using the maximum number of acoustic signals that could be detected in the acoustic wave detection area is used. Thus, assuming that there is an element at a certain position in the receiving area, the aggregate of positions in which the point of interest where that element was extracted is included in the directionality range is calculated as the acoustic wave detection area. The calculated area becomes the area on the scanning surface of the acoustic detector obtained by using the position information in which was subject to coordinate conversion to a point in the imaging area corresponding to the point of interest of the extracted reconfiguration area. This area becomes the receiving area which is required for the reconfiguration at the extracted point of interest.

Here, as the information related to the acoustic signal measurement capacity of the acoustic signal measurement unit 1100, used may be information stored in the main memory 102 or the magnetic disk 103 as described above in S402.

Specifically, examples of the foregoing information include the shape and travel speed of the acoustic detector, element characteristics, and element arrangement on the receiving surface. Moreover, other examples of the foregoing information include the position and area of the scanning surface of the acoustic detector. In addition, another example of the foregoing information is the signal processing capacity of the probe to detect the acoustic waves and output the acoustic signals. In addition, another example of the foregoing information is the irradiation of light of the photoacoustic waves. Accordingly, all information related to the capacity of the measurement of acoustic waves can be included in the acoustic signal measurement capacity.

Note that, as the acoustic wave detection area in the point of interest, the detecting position group of the acoustic waves in the receiving area may also be obtained as the acoustic wave detection area. In the foregoing case, coordinates or intervals or number of the detecting positions (or elements) are calculated.

In step S406, the acoustic wave measuring method determination unit 1003 records the acoustic wave detection area relative to the extracted point of interest. The format of information related to the acoustic wave detection area is represented, for example, as coordinates showing the area on the scanning area of the acoustic detector associated with the position of the point of interest.

In step S407, the acoustic wave measuring method determination unit 1003 determines whether there is any unprocessed point of interest in the reconfiguration area. If there is an unprocessed point of interest, the processing of steps 404 to 407 is repeatedly executed, and such processing is continued until there are no more unprocessed points of interest. When there are no more unprocessed points of interest in the determination of step 407, the routine proceeds to the processing of step 408.

In step S408, the acoustic wave measuring method determination unit 1003 synthesizes information related to the acoustic wave detection area relative to all points of interest in the reconfiguration area. In addition, in order to reconfigure the overall reconfiguration area with the specified image quality, it is necessary to calculate at which area the acoustic waves need to be acquired. When the imaging area and the reconfiguration area coincide, the foregoing area corresponds to the receiving area during the acquisition of acoustic waves. When the imaging area and the reconfiguration area do not coincide, an acoustic wave acquisition area related to an area other than the reconfiguration area is added, and an area including at least an area for acquiring the acoustic waves required for the reconfiguration of the overall reconfiguration area with the specified image quality is generated as the receiving area.

In addition, the acoustic wave measuring method determination unit 1003 determines the control method of the acoustic signal measurement unit 1100 required for acquiring the acoustic waves in the generated receiving area. For example, the scanning method of the acoustic detector, or the control method of light irradiation in the case of photoacoustic waves; that is, the control method for acquiring acoustic waves in the generated receiving area is generated, together with information related to the receiving area, as information related to the acquisition of acoustic waves.

The relation of the scanning area and object on the scanning surface determined by the acoustic wave measuring method determination unit, and the imaging area specified in the object is now explained with reference to FIG. 5.

In FIG. 5, the object 501 is a part of the subject's body, and is sandwiched by holding plates in the imaging apparatus. The scanning surface 502 simultaneously serves as a holding plate and a scanning surface on which the acoustic detector performs scanning. Depending on the thickness of the scanning surface 502, there will be refraction and attenuation of acoustic waves and light, but the explanation thereof is omitted in the ensuing explanation since they do not affect the gist of the explanation of the routine of the present invention.

The holding plate 503 is a plate for holding the object 501. Since the holding plate 503 is not absolute required, the holding plate 503 is omitted in FIG. 3, but the holding plate 503 is preferably used when the object is of a shape such as a human breast or the fixation of the position is unstable. Moreover, even in cases where the object needs to be compressed for improving the imaging accuracy or other reasons, such object may be treated the same as explained in this embodiment. Reference numeral 504 specifies the interval of the holding plates of FIG. 5. Upon specifying the imaging area, the distance of the interval 504 which corresponds to the depth direction of the imaging area of the object may be automatically measured and used. In the foregoing case, the depth size does not need to be specified in the imaging instruction information.

The acoustic detector 505 scans the receiving area on the scanning surface and detects acoustic waves. Here, the acoustic detector is a probe. The total location that is scanned by the acoustic detector corresponds to the receiving area. Reference numeral 506 specifies the height (length) of the scanning area of the acoustic detector. Reference numeral 506 corresponds to the area on the receiving surface corresponding to the receiving area calculated by the acoustic wave measuring method determination unit 1003, and shows the height of the scanning area when viewing, edge-on, the scanning area to be scanned by the acoustic detector 505 as instructed by the acoustic signal measurement unit 1100. Reference numeral 507 is the vertical surface resulting from extending the scanning area vertically to the holding plate. When an area which occupies the face in contact with the scanning surface of the hexahedron of the object specified as the imaging area is used as the scanning surface, reference numeral 507 will coincide with the boundary of the imaging area. Nevertheless, in this embodiment, since the measurement of acoustic signals is for generating the reconstructed image using the maximum number of acoustic signals that could be detected in the directionality range, the scanning area that is determined based on the required receiving area is large, and the vertical surface 507 does not coincide with the imaging area.

The directivity angle 508 of elements is an angle based on the directionality of elements disposed in the acoustic detector 505. Reference numeral 509 shows the area where a reconstructed image having a high image quality can be generated in cases where the acoustic detector 505 detects acoustic waves in the scanning area. In other words, the high image quality area 509 is the acquirable area as an area where the characteristic information can be acquired with the set accuracy. As shown in the diagram, as the distance from the scanning area increases, the area where reconfiguration can be performed with a high image quality will become narrow due to the limitations caused by the angle of the directivity angle 508 of the elements. Thus, the high image quality area 509 is formed in the shape of a truncated pyramid in which the height is the interval 504 of the holding plates, and the lower base is the scanning area of the scanning surface.

The imaging area 510 is the area showing the imaging range specified in the imaging instruction information and which is the target of the present imaging method. The receiving area is calculated so that the reconstructed image of the specified image quality can be obtained with respect to the specified imaging area 510. In other words, the receiving area is determined so that the specified imaging area 510 is at least included in the acquirable area (high image quality area 509) as the area where the image quality target in this embodiment can be reconfigured. Consequently, the valid acoustic wave detection area relative to the deepest and farthest point of interest when viewed from the acoustic detector within the imaging area can be included in the scanning area.

The acoustic signal measurement unit 1100 moves the acoustic detector within the scanning area and causes the acoustic detector perform scanning, and appropriate acoustic wave acquisition can be performed by performing necessary control to the acoustic wave acquisition. Note that, in this embodiment, to calculate the receiving area (scanning area) means to obtain the acoustic wave detection areas relative to the respective points of interest in the imaging area, and totaling the obtained areas.

The acoustic wave measuring method determination unit 1003 sends information related to the acquisition of acoustic waves to the acoustic wave measuring method instruction unit 1004.

In step S409, the acoustic wave measuring method instruction unit 1004 generates the acoustic wave acquisition instruction information based on information related to the acquisition of acoustic waves, and sends the acquired acoustic wave acquisition instruction information to the acoustic signal measurement unit 1100. Note that the acoustic wave acquisition instruction information is configured, for example, from a command or parameter group which instructs the acquisition of acoustic waves to the acoustic signal measurement unit 1100.

According to the foregoing routine, it is possible to implement the routine of the information processing unit 1000 determining the reconfiguring method and the acoustic wave acquiring method and sending such information to the acoustic signal measurement unit 1100.

The method of implementing the foregoing routine based on the respective blocks of the information processing unit 1000 and the acoustic signal measurement unit 1100 was explained above. Nevertheless, in reality, so as long as the functions of the respective blocks can be realized in the imaging apparatus, there is no particular limitation regarding the arrangement or combination of such blocks or the physical configuration thereof. For example, it could be said that the scanning control unit of the present invention is a combination of the control unit 1102, and the acoustic wave measuring method determination unit 1003 in the information processing unit 1000, in addition to the position control means 1106. Moreover, it could be said that the receiving area determination unit of the present invention for obtaining the receiving area corresponds to the acoustic wave measuring method determination unit 1003 in the information processing unit 1000. Moreover, it could be said that the characteristic information acquisition unit of the present invention corresponds to the reconfiguration processing unit 1006. Moreover, it could be said that the reconfiguration area determination means for determining the reconfiguration area as the acquirable area and the processing determination unit for determining the generating method (reconfiguring method) of image data correspond to the reconfiguring method determination unit 1002. Moreover, it could be said that the instruction information acquisition of the present invention for acquiring information of the specified acquisition area and configuration information related to the accuracy corresponds to the imaging instruction information acquisition unit 1001. Accordingly, the method of combining the information processing unit and the acoustic signal measurement unit for realizing the factors configuring the present invention and distributing the resources should be appropriately carried out according to the situation, and the gist of the present invention will not be lost based on such method.

Note that, in the foregoing routine, the acoustic wave measuring method determination unit 1003 calculated the receiving area by converting coordinates in which one point in the reconfiguration area corresponds to one point of the imaging area, and synthesizing the acoustic wave detection area on the scanning surface relative to the respective points of interest. Nevertheless, the present invention can also be implemented with a method where the reconfiguring method determination unit 1002 calculates the receiving area as relative three-dimensional position information relative to the required receiving area and reconfiguration area in advance based on information related to the acquisition of acoustic waves of the acoustic signal measurement unit 1100. In the foregoing case, the acoustic wave measuring method determination unit 1003 converts the reconfiguration area into the coordinate system of the imaging area, and identifies the receiving area on the scanning surface in the imaging process based on the area information which is defined based on the relative position information relative to the reconfiguration area.

Moreover, this embodiment explained the imaging area and the reconfiguration area which normally coincides with the imaging area as a hexahedron. Nevertheless, the present invention can be applied to any type of area so as long as that area can be specified as a three-dimensional area. The imaging area in the foregoing case can be specified by using the mathematical formula of the face to become the boundary of the three-dimensional area or the information group of the range of value, or information of the voxel group associated with the coordinate system of the imaging area.

FIG. 6 is a flowchart showing the routine of the acoustic wave measuring method instruction unit 1004 measuring the acoustic waves related to the specified imaging area, generating acoustic wave reception information, and sending the generated acoustic wave reception information to the information processing unit 1000. The flowchart of FIG. 6 is started when the acoustic wave acquisition instruction information sent from the acoustic wave measuring method instruction unit 1004 of the information processing unit 1000 is received.

In step S601, the acoustic signal measurement unit 1100 controls the acoustic detector and measures the acoustic waves in the receiving area. Control related to the acoustic wave measurement is performed by the control unit 1102 based on the acoustic wave instruction information sent from the information processing unit 1000. In the case of a photoacoustic wave diagnostic apparatus, the irradiation position or timing of light is controlled, measurement of acoustic waves is continued while synchronizing with the position of the acoustic detector or the timing of recording the detected acoustic waves, and the acoustic waves at the respective positions of the receiving area are thereby detected.

When the measurement of acoustic waves is complete, in step S602, the control unit 1102 generates acoustic wave reception information. The acoustic wave reception information is information (receiving condition information) related to the acoustic signals detected at the respective positions in the receiving area and the reception of acoustic waves. If the acoustic signals are detected a plurality of times at the same position, an average value or a representative value may be used. Moreover, correction of the acoustic signals may also be performed at this stage. Information related to the detection of acoustic waves includes information such as acoustic wave acquisition conditions pertaining to the detection of acoustic signals and the determination of acoustic signal values.

In step S603, the acoustic signal measurement unit 1100 sends, to the information processing unit 1000, acoustic wave reception information relative to the receiving area generated by the measurement of acoustic waves.

Based on the foregoing routine, the acoustic signal measurement unit 1100 measures the acoustic wave and sends the acoustic wave reception information to the information processing unit 1000.

FIG. 7 is a flowchart showing the routine of the information processing unit 1000 performing the reconfiguration processing and displaying the imaging data acquired from the imaging method of the present invention based on the acoustic wave reception information sent from the acoustic signal measurement unit 1100. The flowchart of FIG. 7 is started when the acoustic wave reception information acquisition unit 1005 receives the acoustic wave reception information from the acoustic signal measurement unit 1100.

In step S701, when the acoustic wave reception information acquisition unit 1005 acquires the acoustic wave reception information, the acoustic wave reception information acquisition unit 1005 sends the acquired acoustic wave reception information to the reconfiguration processing unit 1006.

In step S702, when the reconfiguration processing unit 1006 receives the acoustic wave reception information from the acoustic wave reception information acquisition unit 1005, the reconfiguration processing unit 1006 performs the reconfiguration processing based on the information related to the reconfiguring method and the information related to the reconfiguration area sent from the reconfiguring method determination unit 1002, and then generates the reconstructed image relative to the imaging area. The reconstructed image corresponds to, for example, the volume data corresponding to the imaging area.

Here, if the reconfiguration area does not coincide with the imaging area, in addition to the reconfiguration area, it is also possible to generate volume data of the imaging area size, and generate volume data in which the voxel value to which the reconfiguration area corresponds is used as the voxel value of the reconstructed image.

The reconfiguration processing unit 1006 sends the generated reconstructed image to the display information generation unit 1007.

In step S703, the display information generation unit 1007 generates display information that can be displayed on the display unit 1008 based on the generated reconstructed image.

As an example of the display image information, there is a method of superimposing and displaying the cross section image of the reconstructed image and the boundary line of the area partitioned based on the favorable or poor image quality on the cross section image such as in cases where the reconstructed image is to be displayed with MPR (Multi Planner Reconstruction). Moreover, the display image may also be displayed via volume rendering. Moreover, in addition to images, information other than images such as textual explanations based on the voxel value of volume data showing the image value; that is, the characteristic information of the respective positions of the three-dimensional reconstructed image showing the characteristic information may also be generated.

The display information generation unit 1007 sends the generated display information to the display unit 1008.

The display unit 1008 is a display device such as a monitor 105. In step S704, the display information generated by the display information generation unit 1007 is displayed, and the information that was imaged based on the imaging method of the present invention is displayed.

According to the foregoing routine, the reconfiguration processing of the information processing unit 1000 in this embodiment and the display of imaged information can be implemented.

Here, while this embodiment explained the receiving area as one area, the receiving area may also be partitioned into a plurality of areas and then specified. For example, a receiving area may be set as one area relative to the overall reconfiguration area, the receiving area may also be partitioned according to the partitioning of the reconfiguration processing, and the acoustic wave reception information may be measured for each receiving area.

Moreover, the acoustic wave measuring method determination unit 1003 may also be configured to be included in the acoustic signal measurement unit 1100. In the foregoing case, the acoustic wave measuring method instruction unit 1004 sends information related to the reconfiguring method and information related to the reconfiguration area to the acoustic signal measurement unit 1100, and processing related to the calculation of the receiving area is executed in the acoustic signal measurement unit 1100.

In addition, the imaging apparatus may also be configured as a result of the information processing unit 1000 and the acoustic signal measurement unit being integrated.

The imaging method of the present invention can be performed as a result of implementing the foregoing routine. Moreover, as a result of being able to implement the imaging method of the present invention according to the foregoing routine, it is possible to perform imaging capable of obtaining a reconstructed image with a uniform image quality that is more appropriate for diagnostic imaging.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-166675, filed on Jul. 29, 2011, which is hereby incorporated by reference herein in its entirety.

1. An object information acquiring apparatus which acquires characteristic information in an object by using an acoustic signal output from an acoustic detector including an element for receiving an acoustic wave which has propagated in the object, comprising:
a receiving area determination unit for obtaining a receiving area of receiving the acoustic wave by using information of a specified acquisition area for the characteristic information specified by a user, configuration information related to accuracy of the characteristic information to be acquired, and receiving condition information on receiving conditions of the acoustic wave; and
a scanning control unit for controlling scanning of the acoustic detector by using information of the receiving area obtained by the receiving area determination unit.

2. The object information acquiring apparatus according to claim 1, further comprising:
an acquirable area determination unit for obtaining an acquirable area, which is an area in which the characteristic information can be acquired with the accuracy set to the specified acquisition area, by using the information of the specified acquisition area, the configuration information related to the accuracy, and the receiving condition information,
wherein the receiving area determination unit obtains the receiving area by using information of the acquirable area.

3. The object information acquiring apparatus according to claim 1, further comprising:
a characteristic information acquisition unit for acquiring characteristic information by using the acoustic signal,
wherein the receiving area determination unit calculates an acoustic wave detection area, which is an area capable of receiving an acoustic wave required for acquiring the characteristic information with the set accuracy for each point of interest contained in the specified acquisition area, and aggregates the acoustic wave detection areas in the respective points of interest to use the aggregated acoustic wave detection areas as the receiving area, and
the characteristic information acquisition unit acquires the characteristic information in each of the points of interest by using the acoustic signal based on the acoustic wave detected from the acoustic wave detection area corresponding to that point of interest.

4. The object information acquiring apparatus according to claim 1,
wherein the receiving condition information includes information related to a position, sensitivity or directionality of the element.

5. The object information acquiring apparatus according to claim 3, wherein the acoustic wave detection area calculated for each point of interest is, when an element is positioned in that acoustic wave detection area, an area in which the point of interest is included in a directionality range of that element.

6. The object information acquiring apparatus according to claim 1, further comprising:
an instruction information acquisition unit for acquiring the information of the specified acquisition area specified by the user, and the configuration information related to the accuracy.

7. The object information acquiring apparatus according to claim 1, further comprising:
a processing determination unit for determining a method of acquiring the characteristic information as image data based on the specified acquisition area, and the configuration information related to the accuracy.

8. The object information acquiring apparatus according to claim 7, wherein the processing determination unit changes the specified acquisition area from the specified range when it is determined that image data of the specified acquisition area cannot be generated with an image quality corresponding to the set accuracy.

9. The object information acquiring apparatus according to claim 1, wherein the acoustic wave which has propagated in the object is a photoacoustic wave which is generated from the object irradiated with light.

10. The object information acquiring apparatus according to claim 1,
wherein the configuration information related to the accuracy is set based on a specification from the user, and
the object information acquiring apparatus further comprises an input unit to be used by the user for specifying the specified acquisition area and the configuration information related to the accuracy.

11. An object information acquiring method by an object information acquiring apparatus which acquires characteristic information in an object by using an acoustic signal output from an acoustic detector including an element for receiving an acoustic wave which has propagated in the object, comprising:
a receiving area determination step of a receiving area determination unit obtaining a receiving area of receiving the acoustic wave by using information of a specified acquisition area for the characteristic information specified by a user, configuration information related to accuracy of the characteristic information to be acquired, and receiving condition information on receiving conditions of the acoustic wave; and
a scanning control step of a scanning control unit controlling scanning of the acoustic detector by using information of the receiving area obtained in the receiving area determination step.

12. The object information acquiring method according to claim 11, further comprising:
an acquirable area determination step of acquirable area determination unit obtaining an acquirable area, which is an area in which the characteristic information can be acquired with the accuracy set to the specified acquisition area, by using the information of the specified acquisition area, the configuration information related to the accuracy, and the receiving condition information,
wherein, in the receiving area determination step, the receiving area is obtained by using information of the acquirable area.

13. The object information acquiring method according to claim 11, further comprising:
a characteristic information acquisition step of a characteristic information acquisition unit acquiring characteristic information by using the acoustic signal,
wherein, in the receiving area determination step, an acoustic wave detection area, which is an area capable of receiving an acoustic wave required for acquiring the characteristic information with the set accuracy, is calculated for each point of interest contained in the specified acquisition area, and the acoustic wave detection areas in the respective points of interest are aggregated to use the aggregated acoustic wave detection areas as the receiving area, and
in the characteristic information acquisition step, the characteristic information is acquired in each of the points of interest by using the acoustic signal based on the acoustic wave detected from the acoustic wave detection area corresponding to that point of interest.

14. The object information acquiring method according to claim 11, wherein the receiving condition information includes information related to a position, sensitivity or directionality of the element.

15. The object information acquiring method according to claim 13, wherein the acoustic wave detection area calculated for each point of interest is, when an element is positioned in that acoustic wave detection area, an area in which the point of interest is included in a directionality range of that element.

16. The object information acquiring method according to claim 11, further comprising:
an instruction information acquisition step of an instruction information acquisition unit acquiring the information of the specified acquisition area specified by the user, and the configuration information related to the accuracy.

17. The object information acquiring method according to claim 11, further comprising:
a processing determination step of a processing determination unit determining a method of acquiring the characteristic information as image data based on the specified acquisition area, and the configuration information related to the accuracy.

18. The object information acquiring method according to claim 17, wherein, in the processing determination step, the specified acquisition area is changed from the specified range when it is determined that image data of the specified acquisition area cannot be generated with an image quality corresponding to the set accuracy.
